(19) United States
(12) Patent Application Publication
(10) Pub. No.: US 2026/0090999 A1
(43) Pub. Date: Apr. 2, 2026

(54) COMPOSITIONS FOR TOPICAL AND TRANSDERMAL DRUG DELIVERY

(71) Applicant: Latitude Pharmaceuticals, Inc., San Diego, CA (US)

(72) Inventors: Andrew Xian Chen, San Diego, CA (US); Norman Keith Orida, San Diego, CA (US); Mohammad Nafiujjaman, San Diego, CA (US); Mohammad Saiful Islam, San Diego, CA (US)

(21) Appl. No.: 19/337,643

(22) Filed: Sep. 23, 2025

Related U.S. Application Data
(60) Provisional application No. 63/701,813, filed on Oct. 1, 2024.

(51) Int. Cl. A61K 9/70; A61K 9/00; A61K 31/00; A61K 31/045; A61K 31/165; A61K 31/192; A61K 31/465; A61K 31/5415; A61K 31/568; A61K 36/81; A61K 47/06; A61K 47/08; A61K 47/10; A61K 47/14; A61K 47/18; A61K 47/20; A61K 47/22; A61K 47/26; A61K 47/32

(52) U.S. Cl. CPC A61K 9/7015; A61K 9/0014; A61K 31/045; A61K 31/165; A61K 31/192; A61K 31/465; A61K 31/5415; A61K 31/568; A61K 31/658; A61K 36/81; A61K 47/06; A61K 47/08; A61K 47/10; A61K 47/14; A61K 47/18; A61K 47/20; A61K 47/22; A61K 47/26; A61K 47/32

(57) ABSTRACT

This invention provides a liquid composition for topical and transdermal drug delivery, comprising a polymethacrylate at >5% weight, a humectant, a plasticizer, and a solvent. Optionally, it includes a permeation enhancer and/or a drug. Upon application and rapid drying, the composition forms a durable, non-sticky, non-transferable film that enables controlled drug release through the skin or retention at the skin surface.

This application claims priority to U.S. Provisional patent application No. 63/701,813, filed Oct. 1, 2024, the contents of which are hereby incorporated by reference in its entirety for all purposes.

### BACKGROUND OF THE INVENTION

Topical formulations, which are applied directly onto the skin in a gel, ointment, cream, patch, or other form, can be useful in pharmaceutical, cosmetic, and personal care applications.

A topical formulation containing a drug allows the drug to stay on the skin surface (e.g., insect repellent), into the skin or intradermally for a localized skin treatment (e.g., acne), or pass through the skin or transdermally. Topical formulations can avoid unwanted systemic exposure and toxicity of a drug and provide better user compliance through greater convenience and ease of use. A topical formulation for a transdermally deliverable drug offers key advantages such as the avoidance of first-pass metabolism, and/or the avoidance of painful and inconvenient injections. Such advantages have prompted the development of transdermal patches, ointments and gels for systemic (whole body) drug delivery.

Patches are a common dosage form for transdermal drug delivery. Examples include drugs for smoking cessation (nicotine patch) and motion sickness (scopolamine patch). However, pre-cut and fixed dose patches are not amenable to dose adjustment. Skin irritation and adhesion issues are well known shortcomings of patches. Furthermore, patches can be an embarrassing presence causing some users to refrain from activities that require some degree of undressing (e.g., swimming, sunbathing, etc.).

Ointment, creams and gels are the most common topical dosage forms. But these are not ideally suitable for transdermal or intradermal drug delivery due to their inability to adhere to the skin for a long period. A regular ointment, cream or gel contains non-volatile oil or a liquid component and will remain as a “wet” or sticky layer after being applied to the skin. Such a wet layer can be easily rubbed off by clothing or other body parts that come in contact with the wet skin area. The inadvertent removal by such clothing or skin contact reduces the amount of the drug and its residency time on the skin and thus results in inconsistencies in drug dosing and activity.

Furthermore, a wet preparation can be readily smeared or transferred from a user to another person by skin contact, potentially resulting in undesired exposure and adverse effects in the recipient (i.e., “secondary exposure”). For drugs such as testosterone and progestin, inadvertent transfer of these highly potent drugs, especially to a prepubescent child, could have highly undesirable consequences. For example, Androgel® is a wet 1% testosterone gel intended for testosterone replacement (a transdermal drug). It causes secondary exposure of testosterone in children or women who had skin contact with the patients. Because of these potentially serious consequences, Androgel® and other wet and potentially transferable testosterone gel products (e.g., Testim® 1%) have FDA-mandated “Black Box” warnings regarding the secondary exposure.

What is needed is a new topical formulation that can be applied to the skin easily like a regular gel, and subsequently dries and remains on the skin like a patch, i.e., cannot be rubbed off or smeared easily, and, more importantly, this in situ formed patch is not “wet” and unlikely to transfer the drug to another individual by skin contact and thereby cause secondary drug exposure. The present disclosure satisfies this need by providing novel compositions referred to as “GelPatch”. Other advantages are also disclosed.

### BRIEF SUMMARY OF THE INVENTION

A primary object of the present invention is to provide a liquid composition that can be easily applied to the skin, and once applied, dries quickly to form a dry and durable film that adheres and conforms to the skin surface. The film resists wear from normal rubbing and contact with clothing or other body parts. A drug, if added, is embedded in the film and is non-transferable by skin contact, therefore avoiding the secondary exposure.

In one aspect of this present invention, the liquid composition is a uniform solution or gel that can be applied by manual spreading on the skin (“rub-on”) or sprayed as aerosol onto the skin (“spray-on”).

In one aspect of this present invention, the liquid composition dries quickly to form a thin, surface-conforming, skin-adherent and dry-feel (or non-sticky) film on the skin in no more than 5 minutes.

A specific object of the present invention is to provide a liquid composition comprising a substantial amount of a polymethacrylate polymer as the film-forming agent.

Another specific object of the present invention is provide a liquid composition containing other functional components, including but not limited to a humectant, a permeation enhancer, a plasticizer, a pH adjuster and a solvent, each in a carefully adjusted concentration range to provide the desired properties for (i) the liquid composition, i.e., rub-on/spray-on, quick-drying, etc. and for (ii) the film composition, i.e., being thin, surface-conforming, skin-adherent, dry-feel/non-sticky, non-peelable, non-transferable, and with the ability to protect the skin or deliver a drug.

In one aspect of this present invention, the film composition can deliver a drug topically (on the skin surface), intradermally (into the skin layer) or transdermally (through the skin into the blood circulation.).

The present invention provides liquid compositions and a method of preparation thereof comprising: a polymethacrylate at a concentration no less than 5% of the composition weight, a humectant, a plasticizer, a pH adjuster and a solvent, and optionally, a permeation enhancer and/or a drug, wherein said composition can be applied to the skin by rubbing or spraying and said composition dries rapidly to form film on the skin.

The present invention also provides a film composition, which is formed from the aforementioned liquid composition but is substantially devoid of the solvent due to evaporation after being applied onto the skin, where said film is thin, non-sticky, skin adherent and abrasion-resistant, and capable of delivering a drug transdermally, intradermally or maintaining the drug on the skin surface.

Both the liquid and the film compositions disclosed herein are made possible by having achieved certain critical balances between some physical factors that are otherwise would work against each other. Some examples are:
1. The liquid composition requires a relatively high polymer content (≥5%) to form a film and yet it maintains a low viscosity and is spray-able.
2. The film composition can adhere to the skin tightly to prevent it from being rubbed off by clothing and can hold the drug tightly to prevent secondary exposure, but at the same time it can also deliver the drug into the skin.
3. The film has a dry feel (non-sticky) and yet it is highly flexible, nonbrittle, non-obtrusive and essentially invisible.
4. The liquid composition contains water as a solvent, so the composition is biocompatible and non-flammable, but the liquid composition is also fast drying-faster than other water-containing preparations.

These and other objects, aspects and embodiments will become more apparent when read with the detailed description and figures that follow.

### BRIEF DESCRIPTION OF THE DRAWINGS

FIG. 1 illustrates a diagram of a Franz cell apparatus.

FIG. 2 illustrates an embodiment of the disclosure, wherein in vitro Franz cell transdermal delivery of ibuprofen through porcine skin by a liquid containing 10% ibuprofen as described in EXAMPLE 2 compared with a commercial product (IbuGel™), which contains 5% ibuprofen in a wet gel formulation.

FIG. 3 illustrates an embodiment of the disclosure, wherein in vitro Franz cell transdermal delivery of ibuprofen through porcine skin by liquid compositions of the present invention containing ibuprofen as described in EXAMPLE 2.

FIG. 4 illustrates an embodiment of the disclosure, wherein in vitro Franz cell transdermal delivery of ibuprofen through porcine skin by a rub-on liquid composition of the containing 10% ibuprofen and 14% polymethacrylate (EXAMPLE 2) and a spray-on formulation containing 10% ibuprofen and 6% polymethacrylate (EXAMPLE 3).

FIG. 5A-B illustrates an embodiment of the disclosure, wherein in vitro transdermal delivery through porcine skin delivered by liquid compositions containing 20% nicotine, 1% testosterone and 1.5% meloxicam (EXAMPLE 4). FIG. 5A shows the amount in mg/sq cm and FIG. 5B shows the % drug applied to the skin delivered through the porcine skin, within the first 50 hours. For example, about 24% (or 2.4 mg) of nicotine applied (10 mg) passed through the skin within 24 hours.

FIG. 6 illustrates an embodiment of the disclosure, wherein in vitro transdermal delivery of a cannabinoid through porcine skin by liquid compositions of the present invention containing different skin permeation enhancers (EXAMPLE 5).

### DETAILED DESCRIPTION OF THE INVENTION

### Definitions

The term “transdermal” as used herein, is a route of administration where drugs are delivered across the skin for systemic (whole body) distribution.

The term “transdermally available drug or transdermally delivered drug” means a drug that can pass through the skin when applied onto the skin or topically, with or without a permeation enhancer.

The term “polymethacrylate”, also known as methacrylic acid copolymer, or ammonio methacrylate copolymer, refers to any fully polymerized copolymer of methacrylic acid and an acrylic or methacrylic ester, including three types, type A (e.g., Eudragit® L by BASF), type B (e.g., Eudragit® S by BASF) and type C (e.g., Eudragit® L 30 D-50 or Eudragit® L 100-55 by BASF), which vary in their methacrylic acid content and solution viscosity. Polymethacrylate also includes two additional polymers, type A (e.g., Eudragit® RL by BASF) and type B (e.g., Eudragit® RS by BASF), which are also referred to as ammonio methacrylate copolymers, consisting of fully polymerized copolymers of acrylic acid and methacrylic acid esters with a low content of quaternary ammonium groups. The typical molecular weight of polymethacrylate is ≥100,000. In pharmaceutical applications, polymethacrylates are generally used in oral capsule and tablet formulations as coating agents such as enteric coatings, sustained release coatings, etc. The preferred polymethacrylate for the present invention is methacrylic acid-ethyl acrylate copolymer (1:1) or Eudragit® L 100-55.

Polymethacrylate is insoluble in water and therefore can stay on the skin for a prolonged or desired period without being affected by perspiration.

It is obvious that a polymer used for a composition of the present invention must be biocompatible with the skin, particularly, it must not cause any irritation, redness, inflammation to the skin. Surprisingly, the inventor of this application has discovered that a polymethacrylate is biocompatible with the skin, despite the facts that it is not conventionally used for topical application and is customarily used for oral drug formulations.

It is also a critical observation that only polymethacrylate, not other polymers, can provide the desired film-forming (discussed later) and drug delivery properties. For example, water-soluble polymers that are commonly used in topical or transdermal drug formulations, such as polyethylene glycols, cellulose polymers (e.g., CMC, HPMC), carbomer, polycarbophil, polyvinyl pyrrolidone, polyethylene alcohol, polyethylene fatty acid ester, polyethylene oxide, polyoxyl 15 hydroxystearate, polyoxyl 20 cetostearyl ether, polyoxyl 35 castor oil, polyoxyl 40 hydrogenated castor oil, polyoxyl 6 and polyoxyl 32 palmitostearte, polypropylene, polypropylene glycol, polypropylene glycol 11 stearyl ether, polysrbate, polyvinyl acetate, polyvinyl alcohol, povidone/eicosane copolymer, and sodium polyacrylate, are not useful for the composition of this invention, because they do not form a durable and perspiration-resistant film.

On the other hand, many other water-insoluble polymers commonly used in topical and transdermal drug formulations, such as 2-ethylhexyl acrylate-vinyl acetate-aluminum acrylate copolymer, acrylates copolymer, acrylic acid-2-ethylhexyl acrylate-2-hydroxyethyl acrylate-vinyl acetate copolymer, acrylic acid-isooctyl acrylate copolymer, aluminum polyester, carbomer copolymer type b (allyl pentaerythritol crosslinked), dimethiconol/trimethylsiloxysilicate crosspolymer, dimethylaminoethyl methacrylate-butyl methacrylate-methyl methacrylate copolymer, ethylene-propylene copolymer, ethylene-vinyl acetate copolymers, polyethylene, polyacrylic acid, polybutene, polyester, polyester polyamine copolymer, polyisobutylene, polyisobutylene/polybutene adhesive, polyvinyl chloride-polyvinyl acetate copolymer, silicone/polyester, trimethylsilyl treated dimethiconol/trimethylsiloxysilicate crosspolymer, isoparaffin/laureth-7/polyacrylamide, carbomer copolymer type b, carbomer homopolymer, carbomer homopolymer type a, cyclomethicone/dimethicone copolyol, and polyethylene glycol monomethyl ether, are too insoluble and therefore cannot be dissolved to form a one-phase rub-on or spray-on solution in the desired and specific solvent (discussed later) used in the composition of the present invention. Moreover, if one of these polymers forms a film on the skin, the resulting film will lock up the drug and prevent its release into the skin (a “dead film”). An example of a dead film is the commercial product Krazy Glue (containing ethyl cyanoacrylate esters), which can be applied to skin, dries rapidly to form a dry film, but the film is unable to deliver a drug.

In one embodiment, a polymethacrylate is added to a liquid composition of the present invention, as a film-former, to a concentration between 5% and 20%, preferably between 6% and 18% and more preferably between 10% and 16% of the composition weight.

It was found by the inventors that when polymethacrylate is added to below 5% of the liquid composition weight, the film formed is too thin and not able to hold enough drug or other functional excipients (humectant, plasticizer, transdermal enhancer etc.) or maintain the film for the desired duration on the skin. Other the other hand, when polymethacrylate is added to more than 20% of the liquid composition weight, the liquid composition is too viscous to spray and the film formed on the skin is too thick, easily peelable, stiff, uncomfortable to wear and is less efficient in delivering a drug.

The term “humectant” refers to a compound that has strong tendency to absorb moisture from its surrounding or is hygroscopic. The purpose of a humectant in the compositions of this invention is to maintain certain water content in the film to maintain its flexibility and to allow for certain mobility of the drug to release from the film into the skin layers (intradermally) or through the skin (transdermally).

Most commonly known humectant excipients (inactive ingredients approved by FDA for use in a drug formulation) can be considered for this purpose. Examples of humectant excipients include sugars, mono- or di-saccharides, polysaccharides, poly alcohols, and polyols. The preferred humectant for this application is glycerol, sorbitol, propylene glycol, or a mixture thereof, because they are readily soluble in the liquid composition of this invention, are biocompatible with the skin and can, with polymethacrylate, form a film on the skin that is transparent, almost invisible, flexible, non-peelable, and durable with a dry feel-even while the film itself contains some water (0.1%-5%). More importantly, a film formed with these humectants can deliver a drug.

It is critically important that a proper concentration of a humectant is used in the compositions of this invention. No or too little (<0.1%) humectant will result in a dry, white, inflexible and brittle film that locks up the drug and prevents its release. On the other hand, too much (>5%) humectant causes the film to lose its dry feel, become sticky to the touch and is uncomfortable to wear. A sticky film is also subject to being easily rubbed off due to contact with clothing or another person, leading potentially to secondary exposure.

In one embodiment, the humectant is selected from a group consisting of glycerol, sorbitol, propylene glycol, or a mixture thereof.

In another embodiment, the humectant is present at a concentration between 0.1% and 5%, preferably between 0.5% and 3% and more preferably between 1% and 2% of the liquid composition weight.

The term “topical” as used herein means, as a route of administration, that the composition is applied to body surfaces such as the skin, nails or mucous membranes.

The term “film” as used herein refers to a thin and solid membrane with a thickness of about 5 to 100 micrometers.

The term “rub-on” as used herein means, a way to apply a topical preparation such as an ointment, cream, gel, etc. by spreading a small amount of the preparation with a finger or with an object, such as a spatula, cotton tip, a brush, or a roll-on ball, on the skin surface. The typical amount of the liquid composition of the present invention to be applied by the rub-on method is about 5 to 20 microliters per square centimeter of the skin area.

The term “spray-on” as used herein means, a way to apply a topical liquid preparation such as lotion or gel, etc. using a spray disperser, such as a pump disperser or aerosol disperser, to deliver a small amount of the liquid preparation as a mist or aerosol over a skin area.

The term “plasticizer” refers to a component added in the composition of the present invention that softens the film and aids in the formation of a flexible, adherent and surface-conforming film on the skin.

In one embodiment, the plasticizer is selected from a group consisting of citrate ester, dimethyl isosorbide, castor oil, propylene glycol, polyethylene glycol and a mixture thereof. The preferred plasticizer is triethyl citrate for a composition of the present invention.

The plasticizer concentration is also an important consideration for a composition of the present invention. No or too little plasticizer results in a film that is brittle, stiff and non-elastic and therefore gives an uncomfortable skin feel and can easily break and peel off. On the other hand, too much plasticizer can cause the film to have a wet feel and is difficult to dry.

In another embodiment, the plasticizer is added to a concentration between 0.1% and 5% of the liquid composition weight, preferably between 0.5% and 3% of the liquid composition weight and more preferably between 1% and 2% of the liquid composition weight.

The term “permeation enhancer” refers to a component used to enhance drug penetration into and through the skin, preferably by temporarily diminishing the impermeability of the skin. Permeation enhancers have also been called “transderaml enhancers”, “penetration enhancers”, “accelerants” and “sorption promoters”. Many drugs do not have the intrinsic property to penetrate or pass through the skin, and therefore are in need a permeation enhancer for their transdermal or intradermal delivery.

In one embodiment, a composition of the present invention contains a permeation enhancer. Examples of permeation enhancers include tweens, sodium lauryl sulfate; menthol; oleic acid, octyl dimethyl para-amino benzoic acid (Padimate 0); mixed esters of capric and caprylic acid; polyhydric alcohols such as propylene glycol or diethylene glycol monoethyl ether, Transcutol®, isopropyl myristate, limonene, ethyl oleate, 1,8-cineole, menthol, farnesol, camphor, eugenol, geraniol or any two or more of the above in combination. The preferred permeation enhancer for the present invention is selected from a group consisting of isopropyl myristate, camphor, geraniol, limonene, farnesol, eugenol, tween 20, tween 80, transcutol, DMSO, linalool, ethyl oleate, cineole and menthol, and a mixture thereof.

The permeation enhancer concentration is also an important consideration for a composition of the present invention. No or too little (<0.1%) permeation enhancer results in poor transdermal or intradermal delivery for a drug. On the other hand, too much (>10%) permeation enhancer can cause the liquid composition to fail to dry to form a film.

A permeation enhancer is generally desired for transdermal delivery of a drug, but it is not essential for the physical properties of the liquid and film compositions of this invention. If the drug is intrinsically transdermally deliverable, e.g., nicotine or nitroglycerin, or no drug is needed for a film, a permeation enhancer is therefore not needed for a composition of this invention.

In another embodiment, the permeation enhancer is added to a concentration between 0.1% and 10%, preferably between 0.5% and 7% and more preferably between 1% and 5% of liquid composition weight.

The term “pH adjuster” refers to an acid or base used to adjust and/or maintain the pH of the liquid composition of the present invention, to obtain a pH where the drug is stable and/or soluble in the liquid composition, or where the film is biocompatible with the skin.

In one embodiment, a composition of the present invention contains a pH adjuster. Examples of pH adjuster include hydrochloric acid, sulfuric acid, acetic acid, citric acid, sodium hydroxide, potassium hydroxide, ammonia, and triethanolamine, meglumine and other organic amines. The preferred pH adjuster is an organic acid or organic base such as citric acid, acetic acid, triethanolamine, meglumine, or a mixture thereof. Inorganic acids such as hydrochloric acid and sulfuric acid can be too strong and therefore irritating to the skin. Inorganic bases such as sodium hydroxide and potassium hydroxide result in an opaque or white film which is less aesthetically desirable.

The amount of pH adjuster used in the liquid composition depends on the acidity or alkalinity of polymethacrylate used, and the drug's solubility and stability in the liquid composition. In general, if the polymethacrylate or drug is acidic, enough of a basic pH adjuster (e.g., triethanolamine, meglumine) is added to adjust to a pH where all components are dissolved, and the drug remains stable. Similarly, if the polymethacrylate or drug is basic, enough of an acidic pH adjuster (e.g., acetic acid, citric acid) is added to adjust to a pH where all components are dissolved, and the drug remains stable. For skin biocompatibility, the preferred pH for a composition of the present invention is between about pH 3 and about pH 8.

In another embodiment, the pH adjuster is present at a concentration between about 0.1% to 5%, preferably 0.5% to 3% and more preferably 1% to 2% of the lipid composition weight.

A pH adjuster is generally desired but is not essential for the physical properties of the liquid and film compositions. If, after all other components (polymer, drug, etc.) are dissolved in the solvent, the composition can reach the desired pH without a pH adjuster, a pH adjuster is not needed for a composition of this invention.

The term “solvent” refers to a volatile solvent or a combination thereof capable of dissolving the drug and all other ingredients in the liquid composition of the present invention.

The purpose of a solvent is to form a one-phase or uniform liquid composition and to allow for rapid drying to form a film on the skin after application, e.g., rub-on or spray-on.

Clearly, a solvent must be safe for the skin, and its vapor phase must be safe to inhale. Polymethacrylates are insoluble in water but soluble in methanol, ethanol, isopropyl alcohol, acetone, ethyl acetate, methylene chloride or 1 N hydrochloric acid. However, only ethanol and isopropyl alcohol are regarded as safe for topical application. Additionally, volatile silicone fluids such as hexamethyldisiloxane and octamethyltrisiloxane, which are inert or non-toxic, may be used as a solvent.

A pure organic solvent such as 100% ethanol is undesirable because it is flammable, very irritating to the skin, and cannot dissolve some of the ingredients necessary for the liquid composition of the present invention (e.g., a water-soluble drug). An ethanol-water mixture is a preferred solvent. Water reduces irritation and flammability of ethanol and helps dissolve hydrophilic ingredients in the formulation. But water alone cannot dissolve polymethacrylate and compositions containing just water dry too slowly.

In one embodiment, a composition of the present invention contains a solvent selected from a group consisting of water, ethanol, isopropanol, a volatile silicone fluid such as hexamethyldisiloxane and octamethyltrisiloxane, or a mixture thereof.

The amount of a solvent used in the liquid composition can be adjusted as long as it is enough to dissolve all components to their desired concentration, dries fast enough (e.g., <5 min) to form a film on the skin and result in a uniform liquid suitable by spray-on or rub-on application.

In another embodiment, the solvent is present at a concentration between 30% and 80% of the composition weight, preferably between 40% and 70% of the composition weight and more preferably between 50% and 60% of the composition weight.

When a water and ethanol mixture is used, the water-to-ethanol mixing ratio is an important consideration for the liquid composition of this invention. For example, if no or too little ethanol is used, the solvent may not be able to dissolve all components, especially polymethacrylate. Also, if a low concentration of ethanol is used, the liquid will dry too slowly (>5 min) to form a film on the skin. Slow drying is inconvenient for users.

In another embodiment, the solvent present in the liquid composition contains a water-ethanol mixture with a water-to-ethanol mixing weight ratio between 2:1 to 1:10, preferably between 1:1 to 1:5 and more preferably between 1:2 to 1:4.

In one embodiment, a liquid composition of the invention comprises a polymethacrylate, a humectant, a plasticizer, and a solvent, where said composition upon being applied onto the skin is capable of rapidly forming a thin, dry feel, surface-conforming, and skin-adherent film.

In one embodiment, a liquid composition of the invention comprises a polymethacrylate at a concentration between 5% and 20%, a humectant at a concentration between 0.1% and 5%, a plasticizer at a concentration between 0.1% and 5%, and a solvent at a concentration between 30 and 80%, with all concentrations based on the liquid composition weight, where said composition upon being applied onto the skin is capable of rapidly forming a thin, dry feel, surface-conforming, and skin-adherent film.

In one embodiment, a liquid composition of the invention comprises a polymethacrylate, a humectant, a plasticizer, a solvent, and, a drug, where said composition upon being applied onto the skin is capable of rapidly forming a dry feel, thin, surface-conforming, and skin-adherent film and delivering the drug transdermally, intradermally or maintaining the drug on the skin surface.

In one embodiment, a liquid composition of the invention comprises a polymethacrylate at a concentration between 5% and 20%, a humectant at a concentration between 0.1% and 5%, a plasticizer at a concentration between 0.1% and 5%, a solvent at a concentration between 30 and 80%, and a drug at a concentration between 0.1% and 25%, with all concentrations based on the liquid composition weight, where said composition upon being applied onto the skin is capable of rapidly forming a thin, dry feel, surface-conforming, and skin-adherent film.

In one embodiment, a liquid composition of the invention comprises a polymethacrylate, a humectant, a plasticizer, a permeation enhancer, a solvent, and, a drug, where said composition upon being applied onto the skin is capable of rapidly forming a thin, dry feel, surface-conforming, and skin-adherent film and delivering said drug transdermally, intradermally or maintaining the drug on the skin surface.

In one embodiment, a liquid composition of the invention comprises a polymethacrylate at a concentration between 5% and 20%, a humectant at a concentration between 0.1% and 5%, a plasticizer at a concentration between 0.1% and 5%, a permeation enhancer at a concentration between 0.1% and 10%, a solvent at a concentration between 30 and 80%, and a drug at a concentration between 0.1% and 25%, with all concentrations based on the liquid composition weight, where said composition upon being applied onto the skin is capable of rapidly forming a thin, dry feel, surface-conforming, and skin-adherent film.

In another embodiment, a liquid composition of the invention further comprises a pH adjuster at a concentration between 0.1% and 5% of liquid composition weight.

In a preferred embodiment, a liquid composition of the invention has a viscosity between 1 and 100 centipoises. If the viscosity of a liquid composition is less than 1 centipoise, it can feel very watery and becomes runny by the rub-on or spray-on application. On the other hand, if the viscosity of a liquid composition is greater than 100 centipoises, it is too viscous for the spray-on application.

In one embodiment, a liquid composition of the invention has a pH between about 3 and about 8.

In one embodiment, a liquid composition of the invention comprises no drug and the film it forms works as a physical barrier to cover or protect the skin.

In one embodiment, a liquid composition of the invention comprises a drug for topical, intradermal and transdermal drug delivery.

In one embodiment, a liquid composition of the invention is applied in a measured dose over a predetermined skin surface area by rub-on or spray-on application.

In one embodiment, the film formed according to this invention can withstand perspiration or gentle washing with water. But when needed, the film can be removed by rubbing vigorously and washing the film-covered skin area with hot water.

In one embodiment, a liquid composition of the present invention forms a film within 1 to 5 minutes after being applied to a skin surface area.

In one embodiment, the liquid composition of the present invention is applied to the skin in an amount of about 5 to 20 microliters per square centimeter of the skin area.

In one embodiment, a liquid composition of the invention forms a film of about 5 to 100 micrometer thickness after being applied to a skin surface area.

In one embodiment, a liquid composition of the invention forms a film that can stay on the skin for between 6 hours and 48 hours.

In one embodiment, a liquid composition of the invention forms a film that resists wear from normal rubbing and contact with clothing or other body parts.

In one embodiment, a liquid composition of the present invention forms a film that is non-transferable to another skin area or another person and eliminates the chance of secondary exposure.

In one embodiment, a liquid composition of the present invention is packaged in a typical container for a topical drug such as bottle, jar, tube, syringe, packet, spray bottle, or bottle with a pump disperser (like a shampoo bottle), for rub-on application.

In one embodiment, a liquid composition of the present invention is packaged in a spray bottle or an aerosol canister. The aerosol canister may be pressurized or contains a propellant at a concentration between 5% and 90% of the total composition weight. An example of a propellant is trichloromonofluoromethane.

In one embodiment, a liquid composition of the invention comprises a drug which would otherwise require frequent dosing, such as 3 times a day, twice a day or once a day.

In one embodiment, a liquid composition of the present invention comprises a drug desired to achieve a long action, between 1 day and 7 days after each application.

In one embodiment, a liquid composition of the invention comprises a drug with potential risk from secondary exposure.

In one embodiment, a liquid composition of the invention comprises a drug desired to achieve steady blood concentration level.

In one embodiment, a liquid composition of the invention comprises a drug which is subject to first pass metabolism.

In one embodiment, a liquid composition of the invention comprises a drug which would otherwise require an injection for its administration.

In one embodiment, a liquid composition of the invention is biocompatible with and can be applied to the skin, mucous membrane, nails or hairs of a human or animal subject.

In one embodiment, a liquid composition of the invention comprises a drug selected from groups consisting of anti-emetic, anti-anginal, anti-inflammatory, anti-fungal, anti-bacterial, anti-viral, anticancer, a steroid, a steroid hormone, a bronchodilator, a drug used to treat osteoporosis, incontinence, antidepressant, anxiolytic, antimigraine, agents used in smoking cessation therapy, antidiarrheals, anticholinergics, anticonvulsants, drugs for mood disorders/obsessive compulsive disorder, antihypertensive, diuretics, anti-obesity, hormonal peptides and analogues, drugs for benign prostatic hyperplasia, urinary retention and erectile dysfunctions, antiparkinson agents such as dopamine agonists and MAO inhibitors, drugs for sleep disorders, and antidiabetic agents.

In a preferred embodiment for a liquid composition of the invention, a systemic drug (i.e. for circulation to the whole body) such as scopolamine, nitroglycerine, clonidine, isosorbide dinitrate, propanolol, timolol maleate, clonazepam, verapamil, meloxicam, diclofenac, naproxen, ibuprofen, ketoprofen, indomethacin, piroxicam, ketorolac, tromethamine, nimesulide, hydrocortisone, dexamethasone, fluocinolone acetonide, betamethasone, estradiol, noethisterone, testosterone, progesterone, salbutamol, bambuterol, salmeterol xinafoate, fluticasone propionate, mometasone furoate, budesonide, beclomethasone dipropionate, sodium cromoglycate, isoprenaline sulphate, alendronic acid, pamidronic acid, etidronic acid, vasopressin, oxybutynin, imipramine, mirtazapine, desipramine, naratriptan, zolmitriptan, sumatriptan, loperamide, misoprostol, hyoscyamine, atropine and trihexyphenidyl, lorazepam, diazepam, tiagabine, fluoxetine, paroxetine, lisinopril, trandolapril, captopril, amlodipine, felodipine, prazosin, amiloride, methamphetamine, sibutramine, tamsulosin, terazosin, finasteride, alprostadil, sildenafil, bromocriptine, cabergoline, selegiline, melatonin, sulphonyl ureas, glimepiride, rosiglitazone, glyburide, glipizide, or a pharmaceutical acceptable salt, ester or chiral form, or a combination of two or three drugs selected from the list mentioned above, can be used to make the composition of the present invention.

In another preferred embodiment, the composition of the present invention can also contain a topical drug (i.e., for a localized skin condition) including a local anesthetic, antibiotic, antifungal, antiacne agent, chemotherapy agent, steroid, decongestant, fluoride, bleaching agent, antiseptic, hair remover, hair growth promoter, sunscreen, or insect repellent, etc.

In another preferred embodiment, the composition of the present invention can also be utilized, without any drug, to obtain a protective film adhering on the skin surface to protect the skin from UV or sunlight, dryness, contact dermatitis, skin allergen such as poison ivy or poison oak, secondary drug exposure, or bacterial or viral infection.

In yet another embodiment, the composition of the present invention can also contain a colorant, dye, deodorant, anti-perspirant, etc. for cosmetic or personal care uses.

In another embodiment, the present compositions are a significant advance over conventional ointment, cream or gel compositions, as they provide a prolonged adherence to the skin, resistance to abrasion from contact with clothing or other body parts, low potential for secondary exposure, and a high transdermal efficiency for certain transdermally deliverable drugs.

In another embodiment, the present compositions are a significant advance over conventional patch formulations, since they are convenient to apply by either the rub-on or spray-on method, permit easily adjustable dosing, are easy to remove (by simply washing with hot water), are visually more appealing, and are essentially invisible.

The liquid compositions of the present invention are generally prepared by mixing the ingredients at a temperature from about 5 to 60 degrees C. in the solvent until all solid ingredients are dissolved to form a uniform solution, emulsion or dispersion. Alternatively, a liquid composition may be prepared first by combining and mixing all inactive ingredients until all solids are dissolved to forma vehicle, then adding the drug to the already prepared vehicle and mixing until the drug is dissolved.

The following nonlimiting examples illustrate the formulations, testing, characterization and use of compositions of the invention.

### Example 1

A liquid composition of the present invention without a drug (F1) having the composition below was prepared in a 150 g quantity (batch size):

| Component | Function | Supplier | Grade | % w/w | g/150 g. |
|---|---|---|---|---|---|
| Polymethacrylate | Film former | Rohm | USP/NF | 16.00 | 24.0 |
| (Methacrylic Acid- | | | | | |
| Ethyl Acrylate | | | | | |
| Copolymer (1:1)) | | | | | |
| Triethanolamine | pH adjuster | Dow Chemicals | ACC | 1.20 | 1.8 |
| Isopropyl myristate | Permeation | Spectrum | NF | 5.00 | 7.5 |
| | enhancer | Chemicals | | | |
| Glycerol | Humectant | Fisher Scientific | USP | 5.00 | 7.5 |
| Triethyl Citrate | Plasticizer | Spectrum | FCC | 4.20 | 6.3 |
| | | Chemicals | | | |
| Ethanol (Alcohol | Solvent | Spectrum | USP | 50.00 | 75.0 |
| Anhydrous) | | Chemicals | | | |
| DI-water | Solvent | | | 18.6 | 27.9 (or |
| | | | | | add to or |
| | | | | | QS to 150 |
| | | | | | g) |

Water-to-ethanol weight ratio=1:4.4
The following procedure was used to prepare F1:
1. Record tare weight of a 250 mL glass container.
2. Add all components into the container. Record weight of each.
3. Set up a high shear mixer (Silverson Model L5M).
4. Apply a moderate mixing speed (1000-2000 RPM) to allow all solids to completely dissolve to form a uniform translucent and slightly viscous (gel-like) liquid.
5. Fill the liquid in plastic bottles with a pump head/dispensers.
6. Store the bottles at room temperature.

The liquid was readily dispensed by the pump head with each push delivering 0.1 mL-5 mL. The volume can be adjusted by the design of the pump head. The dispensed liquid was applied by a rub-on method using a finger, a spatula, a brush, or a roll-on ball, onto the human skin to form a uniform layer which dried in about 1 minute. The liquid could be applied to almost any intact skin areas including nails and hair.

The film formed was clear (transparent) and hardly visible. It had dry feel (not sticky) and no odor. It conformed to the skin surface and remained on the skin for about 6 hours in the skin areas with high friction with clothing (e.g., the elbows) or about 48 hours in the skin areas with less friction (e.g., the back). The film was flexible, and the wearer could hardly feel it. The film was not peelable, however, with hot water and vigorous rubbing, it came off the skin.

### Example 2

Liquid compositions of the present invention containing the anti-inflammatory drug ibuprofen at 5%, 10%, 15% and 20% having the compositions below were prepared for transdermal delivery:

| Component/Concentration (w/w %) | F2 | F3 | F4 | F5 |
|---|---|---|---|---|
| Ibuprofen | 5.0 | 10.0 | 15.0 | 20.0 |
| Methacrylic Acid-Ethyl Acrylate | 15.2 | 14.4 | 13.8 | 12.8 |
| Copolymer (1:1) | | | | |
| Triethanolamine | 1.1 | 1.1 | 1.0 | 1.0 |
| Isopropyl myristate | 4.8 | 4.5 | 4.3 | 4.0 |
| Glycerol | 4.8 | 4.5 | 4.3 | 4.0 |
| Triethyl Citrate | 4.0 | 3.8 | 3.6 | 3.4 |
| Alcohol Anhydrous | 47.5 | 45.0 | 43.0 | 40.0 |
| DI-water | 17.7 | 16.7 | 15.0 | 14.9 |
| Total | 100 | 100 | 100 | 100 |

Water-to-ethanol weight ratio=1:2.7

The following procedure was used to prepare each of the above compositions:
1. Weigh ibuprofen into a plastic tube.
2. Add F1 (used as a vehicle, prepared in EXAMPLE 1) in the calculated amount to obtain the final concentration of each component as in the above composition table.
3. Shake to dissolve ibuprofen. Heat to about 60 deg C. as needed to accelerate the dissolution of ibuprofen. The 5% and 10% ibuprofen compositions obtained were translucent and the 15 and 20% were clear.

All compositions provided the desired film-forming properties, i.e., fast drying, dry feel, invisible, skin-adherent, skin-conforming, abrasion-resistant and lasted on the skin for 6-48 hours, by a rub-on application.

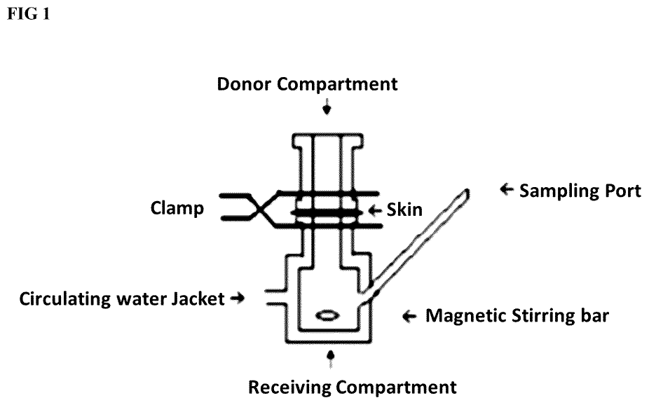

Transdermal delivery of ibuprofen by these compositions were tested using a Franz cell apparatus (FIG. 1) and porcine skin. Franz cell apparatus is a conventional device for measuring transdermal and intradermal drug delivery. Porcine skin has certain anatomical similarity to the human skin and is well-acceptable for in vitro transdermal/intradermal drug delivery testing.

Fresh porcine abdomenal skin was trimmed to remove the fat under the skin to about the same thickness using a surgical instrument called dermatome and then cut into a disc size about 1 inch diameter to fit to a Franz diffusion cell. The skin disc was sandwiched between a donor (top) and receiving chamber (bottom) of the apparatus (FIG. 1) with the skin surface facing the donor chamber. Phosphate buffer saline (PBS) was added in both donor and receiving chambers. Both chambers were maintained at 37° C. The skin was hydrated in PBS for 4 hours. From the donor chamber, 1 mL PBS was removed and replaced with 1 mL of a formulation (e.g., F2). The donor chamber was kept open to allow evaporation of ethanol and form a thin film on the skin. At each sampling point, one milliliter of sample was collected from the receiving chamber. One milliliter of fresh PBS was added to the receiving chamber after each collection. Each collected sample was filtered using a 35 μm filter. Each filtered sample was dilute 2× with methanol and tested by HPLC to determine drug (ibuprofen) concertation in the receiving chamber. From the drug concentration, the amount of drug passed through the skin was calculated. From the amount of drug passed through the skin, the % drug passed through the skin was calculated. Both the amount or % of drug passed through the skin can be used to quantitate drug transdermal delivery efficacy by a formulation and to compare different formulations for transdermal delivery.

For intradermal delivery, after the final sample was taken, the skin was collected, homogenized, and extracted using an organic solvent (e.g., PBS or hexane) for the drug delivered into the skin. The extracted sample was subject to the same HPLC analysis to determine the drug concentration in the skin and to quantitate the intradermal drug delivery.

The F2-F5 liquid formulations contained 5-20% ibuprofen easily applied to the skin's surface using a spatula. After setting the in vitro Franz cells, the F2-F5 formulations were applied to the porcine skin, forming a transparent film that was hardly visible.

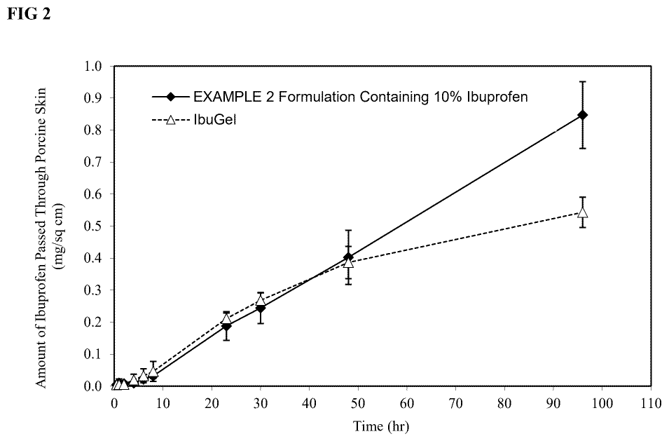

FIG. 2 exhibits transdermal delivery of ibuprofen by an EXAMPLE 2 composition (F3) compared to a commercial ibuprofen topical transdermal drug (IbuGel™). In the first 48 hours, both compositions delivered ibuprofen at comparable rates. However, after 48 hours, the F3 composition maintained its delivery rate while the delivery rate of IbuGel™ noticeably slowed. It is worth noting that IbuGel™ is a “wet” gel, lacking the film forming properties. The composition of the present invention provided greater and longer transdermal delivery than a wet and non-film forming topical transdermal formulation.

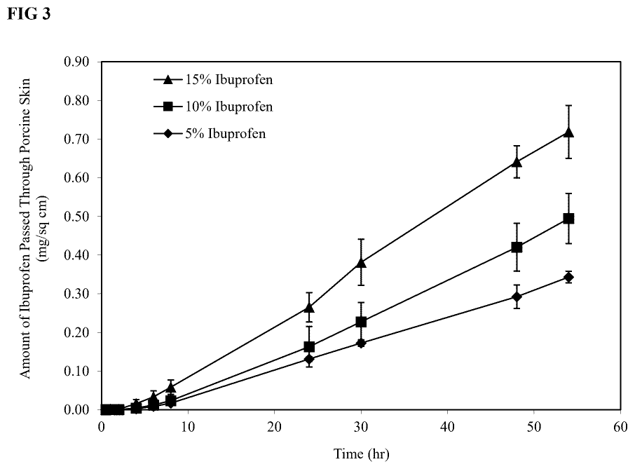

FIG. 3 illustrates the transdermal delivery of ibuprofen using the EXAMPLE 2 composition at three different strengths (5%, 10%, and 15% wt). The findings indicate a clear dose-response correlation between the rate of transdermal delivery (mg/hr) and the ibuprofen concentration in the liquid composition. This observation indicated that a drug concentration in a composition of the present invention can be as high as 20%-which is near the ibuprofen solubility limit in F1.

### Example 3

A spray-on composition of the present invention having the following composition was prepared for transdermal delivery using the same procedure as described in EXAMPLE 2:

| Component | F6 (% w/w) |
|---|---|
| Ibuprofen | 10 |
| Methacrylic Acid-Ethyl | 6 |
| Acrylate Copolymer(1:1) | |
| Triethanolamine | 0.45 |
| Isopropyl myristate | 4.5 |
| Glycerol | 4.5 |
| Triethyl Citrate | 1.575 |
| Alcohol Anhydrous | 55 |
| DI-water, add about | 13.98 |
| Total | 100 |

Water-to-ethanol weight ratio=1:3.9

This liquid composition was a transparent solution having a viscosity of approximately 14 centipoises at 25° C. It was easily sprayed using a conventional topical spray pump (Pfeiffer topical spray pump SAP #62655+34473) into an aerosol. The aerosol deposited on the human skin as a thin layer which dried in about 2 minutes to form a thin film. The liquid was also readily applicable by a rub-on method using a finger or a spatula onto the skin to form a uniform layer which dried in about 1 minute. It could be applied to almost any intact skin areas including nails.

The film formed was clear (transparent) and hardly visible. It had dry feel (not sticky) and no order. It conformed to the skin surface and remained on the skin for about 6 hours in the skin areas with high friction with clothing (e.g., the elbows) and about 48 hours in the skin areas with less friction (the back). The film was flexible, and the wearer could hardly feel it. The film was not peelable, however, with hot water and vigorous rubbing, it came off the skin.

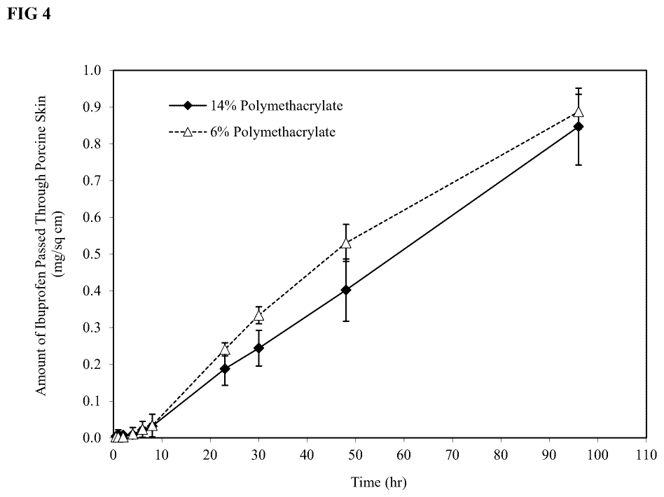

FIG. 4 depicts how the concentration of polymethacrylate in the compositions of the current invention affects the transdermal delivery rate of ibuprofen. Both concentrations provided efficient transdermal delivery of ibuprofen, and the concentration difference between 6% and 14% polymethacrylate had minimal impact on the transdermal delivery rate of ibuprofen. The liquid composition containing 14% polymethacrylate composition is preferred for rub-on application because it is not runny and had a nice smooth gel feel, while the 6% polymethacrylate composition is preferred for spray-on use because it is less viscous.

When polymethacrylate is below 5%, the liquid composition was too runny for the rub-on application, and the film formed was too thin or fragile. When polymethacrylate is above 20%, the liquid composition was too thick and difficult for the rub-on or spray on application.

The transdermal delivery data from this example, along with the viscosity, rub-on and spray on observations, indicated that the polymethacrylate concentration in a composition of the present should be between 5% and 20% of the composition weight.

### Example 4

Liquid compositions of the present invention containing testosterone, nicotine, meloxicam, capsicum oleoresin, capsaicin, menthol and a combination of capsaicin and menthol for transdermal and intradermal delivery were prepared according to the following compositions using the same procedure as described in EXAMPLE 2:

| Component/ | | | | | | |
|---|---|---|---|---|---|---|
| concentration | | | | | | |
| (% w/w) | F7 | F8 | F9 | F10 | F11 | F12 |
| Testosterone | 1.0 | | | | | |
| Nicotine | | 20.0 | | | | |
| Meloxicam | | | 1.5 | | | |
| Capsicum Oleoresin | | | | 10.0 | | |
| Menthol | | | | | 10.0 | 10 |
| Capsaicin | | | | | | 0.15 |
| Methacrylic Acid- | 15.8 | 12.8 | 15.8 | 14.4 | 14.4 | 14.38 |
| Ethyl Acrylate | | | | | | |
| Copolymer (1:1) | | | | | | |
| Triethanolamine | 1.2 | 1.0 | 1.2 | 1.1 | 1.08 | 1.08 |
| Isopropyl myristate | 5.0 | 4.0 | 4.9 | 4.5 | 4.5 | 4.49 |
| Glycerol | 5.0 | 4.0 | 4.9 | 4.5 | 4.5 | 4.49 |
| Triethyl Citrate | 4.2 | 3.4 | 4.1 | 3.8 | 3.78 | 3.77 |
| Alcohol Anhydrous | 49.5 | 40.0 | 49.3 | 45.0 | 45 | 44.93 |
| DI-water, add about | 18.4 | 14.9 | 18.3 | 16.7 | 16.74 | 16.71 |
| Total | 100 | 100 | 100 | 100 | 100 | 100 |
| Intended delivery | Transdermal | Transdermal | Transdermal | Intradermal | Intradermal | Intradermal |
| Indication | Hormone | Smoking | Systemic | Local pain | Local pain | Local pain |
| | replacement | cessation | pain relief | relief | relief | relief |

Water-to-ethanol weight ratio=1:2.7

Each composition was tested for their film-forming properties. All compositions provided the desired film-forming properties, i.e., fast drying, dry feel, invisible, skin-adherent, skin-conforming, abrasion-resistant and lasted on the skin for 6-48 hours, by a rub-on application. For F7, F8 and F9, Franz cell test was performed to evaluate their transdermal drug delivery using the same method as described in EXAMPLE 2.

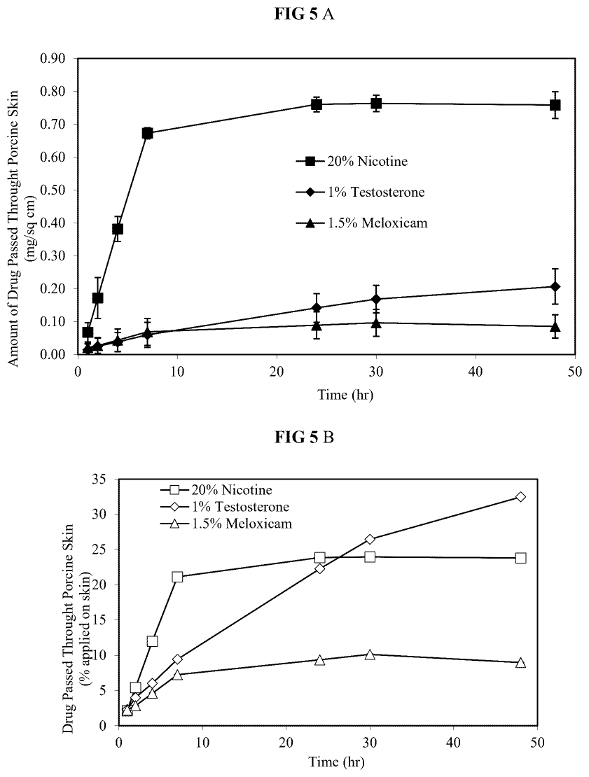

FIG. 5 demonstrates versatility of the compositions of the present invention for transdermal delivery of a variety of drugs. The drugs and drug combination included in the Example 4 varied greatly in their structures, physical properties and indications.

The testosterone composition (F7) delivered about 22% (or 0.45 mg) of the drug applied to the skin (2 mg) in the about 24 hours, which appeared to be substantially higher than Androgel® (about 4% delivery). Androgel® is a commercial drug containing 1% testosterone and was tested transdermal delivery under a similar Franz cell condition (J Pharm Pharmaceut Sci, 13(2)218-230, 2010). Moreover, F7 forms a film and does present any risk of secondary exposure. Clearly, the testosterone composition disclosed herein can be used for hormone replacement therapy.

FIG. 5 also shows the clear transdermal delivery for nicotine and meloxicam by the compositions of the present invention. For nicotine, the amount delivered within the first 24 hours was about 24% (or 2.4 mg) of nicotine applied (10 mg). For meloxicam, the amount delivered was 9% (or 0.28 mg) of the meloxicam applied (3 mg). The nicotine and meloxicam compositions disclosed herein have been contemplated for smoking cessation and anti-inflammatory indications, respectively.

For F10, F11 and F12, human subjects (two inventors) were used for their intradermal or efficacy testing. Each composition was applied by a rub-on method to the human subject in two areas: one on the elbow area (a high friction area with clothing) and one on the back (less friction). It was reported that there was an immediate heating sensation after applying F10 (capsicum oleoresin) and F12 (capsaicin), and the sensation last for about 2 hours. However, upon perspiration or moisture exposure, the heating sensation returned after about 6 hours in the elbow area and about 48 hours on the back. F11 (menthol) had the similar effect but with a cooling sensation.

This finding in this Example 4 suggests that the compositions of the present invention can be used to deliver different drugs by both transdermal and intradermal routes. Their transdermal delivery is comparable or better that some commercial drugs and their intradermal delivery can last for about 6 hours and 48 hours.

### Example 5

Liquid compositions of the present invention containing different permeation enhancers were prepared according to the following compositions using the same method as described in Example 2, for transdermal delivery of a cannabinoid:

| Component | | | | | | | | | |
|---|---|---|---|---|---|---|---|---|---|
| (% w/w) | F13 | F14 | F15 | F16 | F17 | F18 | F19 | F20 | F21 |
| Cannabinoid | 1 | 1 | 1 | 1 | 1 | 1 | 1 | 1 | 1 |
| Limonene | | 10 | | | | | | | |
| Ethyl Oleate | | | 0.2 | | | | | | |
| Cineole | | | | 5 | | | | | |
| Menthol | | | | | 5 | | | | |
| Farnesol | | | | | | 3 | | | |
| Camphor | | | | | | | 5 | | |
| Eugenol | | | | | | | | 5 | |
| Geraniol | | | | | | | | | 5 |
| Methacrylic Acid-Ethyl | 15.8 | 14.2 | 15.8 | 15.0 | 15.0 | 15.4 | 15.0 | 15.0 | 15.0 |
| Acrylate Copolymer(1:1) | | | | | | | | | |
| Triethanolamine | 1.2 | 1.1 | 1.2 | 1.1 | 1.1 | 1.2 | 1.1 | 1.1 | 1.1 |
| Isopropyl myristate | 5.0 | 4.5 | 4.9 | 4.7 | 4.7 | 4.8 | 4.7 | 4.7 | 4.7 |
| Glycerol | 5.0 | 4.5 | 4.9 | 4.7 | 4.7 | 4.8 | 4.7 | 4.7 | 4.7 |
| Triethyl Citrate | 4.2 | 3.7 | 4.1 | 3.9 | 3.9 | 4.0 | 3.9 | 3.9 | 3.9 |
| Alcohol Anhydrous | 49.5 | 44.5 | 49.4 | 47.0 | 47.0 | 48.0 | 47.0 | 47.0 | 47.0 |
| DI-water, add about | 18.4 | 16.6 | 18.4 | 17.5 | 17.5 | 17.9 | 17.5 | 17.5 | 17.5 |
| Total | 100 | 100 | 100 | 100 | 100 | 100 | 100 | 100 | 100 |

Water-to-ethanol weight ratio=1:2.7

Each composition was tested for their film-forming properties. All compositions provided the desired film-forming properties, i.e., fast drying, dry feel, invisible, skin-adherent, skin-conforming, abrasion-resistant and suitable for the rub-on application.

Franz cell test was performed for all above compositions to evaluate their transdermal drug delivery using the same method as described in EXAMPLE 2.

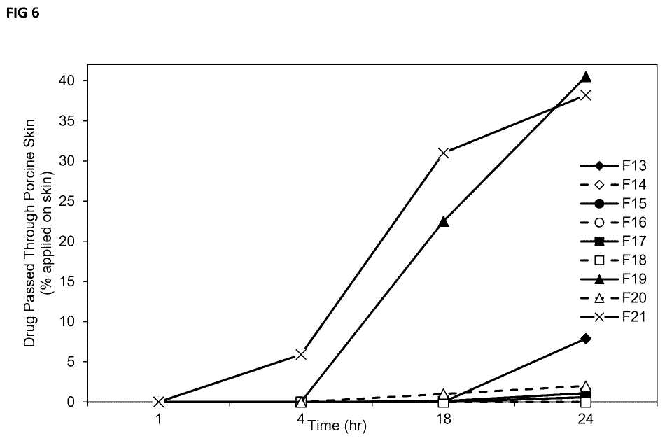

FIG. 6 shows the transdermal delivery of cannabinoid using the composition described in Example 5, with nine different permeation enhancers. The findings indicate that after 24 hours, approximately 40% of cannabinoid was passed through full-thickness porcine skin when a 5% concentration of camphor (F19) was employed as a permeation enhancer. Furthermore, around 38% of the drug passed through the porcine skin in the same timeframe when 5% geraniol (F21) was as a permeation enhancer.

The amounts of cannabinoid (%) passed through porcine skin are provided as following:

| Time | | | | | | | | | |
|---|---|---|---|---|---|---|---|---|---|
| (hr) | F13 | F14 | F15 | F16 | F17 | F18 | F19 | F20 | F21 |
| 1 | 0 | 0 | 0 | 0 | 0 | 0 | 0 | 0 | 0 |
| 4 | 0 | 0 | 0 | 0 | 0 | 0 | 0 | 0 | 5.9 |
| 18 | 0 | 0 | 0 | 0 | 0.1 | 0 | 22.5 | 1 | 31 |
| 24 | 7.9 | 0.6 | 0.6 | 0 | 1.1 | 0 | 40.5 | 2 | 38.2 |

Based on the transdermal delivery test results from this Example 5, a list of preferred permeation enhancers is selected to include isopropyl myristate (in F13), camphor (in F19), geraniol (in F21), limonene (in F14), ethyl oleate (in F15), menthol (in F17), and eugenol (in F20).

### Example 6

Liquid compositions of the present invention containing two drugs were prepared in the F1 vehicle, each with a different permeation enhancer of total 10, using the same method as described in Example 2, for transdermal and intradermal delivery of Drug A and Drug B. The permeation enhancer used include the following:

| Component (% w/w) | F22 | F23 | F24 | F25 | F26 | F27 | F28 | F29 | F30 | F31 |
|---|---|---|---|---|---|---|---|---|---|---|
| Drug A | 5 | 5 | 5 | 5 | 5 | 5 | 5 | 5 | 5 | 5 |
| Drug B | 10 | 10 | 10 | 10 | 10 | 10 | 10 | 10 | 10 | 10 |
| Limonene | 10 | | | | | | | | | |
| Ethyl Oleate | | 0.2 | | | | 2 | | | | |
| 1,8-Cineole | | | 5 | | | | | | | |
| L-Menthol | | | | 5 | | | | | | |
| Linalool | | | | | 5 | | | | | |
| Transcutol HP) | | | | | | | 10 | | | |
| Tween 20 | | | | | | | | 5 | | |
| DMSO | | | | | | | | | 10 | |
| Tween 80 | | | | | | | | | | 5 |
| Polymethacrylate | 12.0 | 13.6 | 12.8 | 12.8 | 12.8 | 13.3 | 12.0 | 12.8 | 12.0 | 12.8 |
| (Methacrylic | | | | | | | | | | |
| Acid-Ethyl Acrylate | | | | | | | | | | |
| Copolymer (1:1)) | | | | | | | | | | |
| Triethanolamine | 0.9 | 1.0 | 1.0 | 1.0 | 1.0 | 1.0 | 0.9 | 1.0 | 0.9 | 1.0 |
| Isopropyl myristate | 3.8 | 4.2 | 4.0 | 4.0 | 4.0 | 4.2 | 3.8 | 4.0 | 3.8 | 4.0 |
| Glycerol | 3.8 | 4.2 | 4.0 | 4.0 | 4.0 | 4.2 | 3.8 | 4.0 | 3.8 | 4.0 |
| Triethyl Citrate | 3.2 | 3.6 | 3.4 | 3.4 | 3.4 | 3.5 | 3.2 | 3.4 | 3.2 | 3.4 |
| Ethanol (Alcohol | 37.5 | 42.4 | 40.0 | 40.0 | 40.0 | 41.5 | 37.5 | 40.0 | 37.5 | 40.0 |
| Anhydrous) | | | | | | | | | | |
| DI-water | 14.0 | 15.8 | 14.9 | 14.9 | 14.9 | 15.4 | 14.0 | 14.9 | 14.0 | 14.9 |
| Total | 100 | 100 | 100 | 100 | 100 | 100 | 100 | 100 | 100 | 100 |

Water-to-ethanol weight ratio=1:2.7

Drug A & B are different in structure and physicochemical properties, but both are intended for transdermal and intradermal delivery for a localized and systemic actions. Franz cell test was performed to evaluate both transdermal and intradermal drug delivery using the same method as described in EXAMPLE 2. For intradermal delivery measurement, the porcine skin was collected after 24 hours, homogenized in PBS. The homogeneity was centrifuged, the supernatant was collected and passed through a 35-micron filter, and finally, tested by HPLC for Drug A and Drug B concentrations.

Transdermal delivery data (amount of Drug (mg/cm2) passed through porcine skin within 24 hours) are listed below:

| | Permeation | Drug | Drug | Rank |
|---|---|---|---|---|
| Composition | enhancer | A | B | Order |
| F22 | Limonene | 0.7 | 0.5 | 9 |
| F23 | Ethyl Oleate | 0.7 | 0.9 | 5 |
| F24 | Cineole | 0.7 | 0.8 | 6 |
| F25 | Menthol | 0.6 | 0.6 | 10 |
| F26 | Linalool | 0.7 | 0.7 | 7 |
| F27 | Transcutol | 3.2 | 4.8 | 3 |
| F28 | Tween 20 | 3.3 | 4.9 | 2 |
| F29 | DMSO | 3.4 | 5.1 | 1 |
| F30 | Tween 80 | 3.1 | 4.8 | 4 |
| F31 | Limonene | 0.7 | 0.5 | 8 |

Intradermal delivery data (mg of drug passed into the skin within 24 hr) are summarized below:

| | Permeation | Drug | Drug | Rank |
|---|---|---|---|---|
| Composition | enhancer | A | B | Order |
| F22 | Limonene | 1.87 | 0.31 | 8 |
| F23 | Ethyl Oleate | 1.90 | 0.25 | 7 |
| F24 | Cineole | 1.75 | 0.34 | 9 |
| F25 | Menthol | 1.43 | 0.28 | 10 |
| F26 | Linalool | 2.00 | 0.37 | 6 |
| F27 | Transcutol | 17.13 | 15.39 | 3 |
| F28 | Tween 20 | 23.55 | 22.49 | 1 |
| F29 | DMSO | 14.85 | 13.77 | 4 |
| F30 | Tween 80 | 17.74 | 15.80 | 2 |
| F31 | Limonene | 11.84 | 11.49 | 5 |

The results indicate all tested permeation enhancer enabled the transdermal and intradermal delivery of Drug A and B concurrently or simultaneously. A permeation enhancers seemed to promote the transdermal and intradermal delivery for both drugs in the same way, suggesting that the permeation enhancers worked by changing the skin permeability rather than the drugs. Therefore, it is meaningful to name a list of preferred permeation enhancers for the liquid compositions for the present invention, since such permeation enhancers may be applied to many other drugs.

The preferred permeation enhancers selected from both Example 5 and Example 6 include isopropyl myristate, camphor, geraniol, limonene, eugenol, tween 20, tween 80, transcutol, DMSO, linalool, ethyl oleate, cineole and menthol.

The permeation enhancer concentration to be used in a composition of the present invention can be as low as 0.2% and as high as 10%.

### Example 7

Stability of Ibuprofen in the EXAMPLE 3 composition were tested by measuring the assay and purity of ibuprofen in F6 composition (at 3 different pH) using an RP-HPLC method with the following conditions:

| Column | Luna 5 μm, C18 (2), 150 × 4.6 mm | |
|---|---|---|
| Mobile Phase A (MPA) | Water, phosphoric acid added to pH 2.5 | |
| Mobile Phase B (MPB) | Acetonitrile | |
| Diluent | 40:60 (V:V) = MPA:MPB | |
| Flow rate | 1.5 | mL/min |
| Column temp. | 65 | deg C |
| UV detection | 214 and 254 nm | |
| Inj. vol. | 10 | μL |
| Standard con. | 1.2 | mg/mL |

| | |
|---|---|
| Elusion | 48% (v/v) MPB and 52% (v/v) MPB |

Stability tested results are listed below:

| | | Assay | Assay recovery | Purity (% |
|---|---|---|---|---|
| Sample ID | Storage | (% w/w) | over T0 (%) | peak area) |
| pH 5.40 | Time 0 | 10.36 | 100 | 99.93 |
| | 25° C./1 wk | 10.35 | 99.9 | 99.94 |
| | 25° C./2 wk | 10.38 | 99.1 | 99.90 |
| | 40° C./1 wk | 10.26 | 99.0 | 99.88 |
| | 40° C./2 wk | 10.37 | 98.9 | 99.83 |
| | 60° C./1 wk | 10.13 | 97.8 | 99.77 |
| | 60° C./2 wk | 10.01 | 95.5 | 99.60 |
| pH 5.85 | Time 0 | 10.39 | 100 | 99.92 |
| | 25° C./1 wk | 10.26 | 99.0 | 99.92 |
| | 25° C./2 wk | 10.32 | 98.3 | 99.86 |
| | 40° C./1 wk | 10.17 | 98.1 | 99.88 |
| | 40° C./2 wk | 10.33 | 98.4 | 99.83 |
| | 60° C./1 wk | 10.12 | 97.7 | 99.74 |
| | 60° C./2 wk | 10.05 | 95.8 | 99.64 |
| pH 6.16 | Time 0 | 10.18 | 100 | 99.90 |
| | 25° C./1 wk | 10.29 | 101.0 | 99.93 |
| | 25° C./2 wk | 10.20 | 100.2 | 99.90 |
| | 40° C./1 wk | 10.04 | 98.6 | 99.88 |
| | 40° C./2 wk | 10.26 | 100.8 | 99.83 |
| | 60° C./1 wk | 10.05 | 98.7 | 99.77 |
| | 60° C./2 wk | 9.95 | 97.8 | 99.59 |

The results show that ibuprofen is very stable in the liquid composition of the present invention (F6) at pH 5.4. pH 5.9 and pH 6.2.

### Example 8

A skin residency or abrasion-resistant test was performed for F2 (5% ibuprofen) composition in EXAMPLE 2. F2 was applied by manual rubbing on the skin surface of the left shoulder and the left knee of a male human volunteer. Approximately 0.3 g of the composition containing 15 mg of ibuprofen was applied over a 6 cm diameter circular area at each location. For comparison, the right shoulder and knee received the same amount of IbuGel®, which is a commercial drug in a wet gel (does not form a film) formulation that contains 5% ibuprofen. The compositions were allowed to dry for 2 minutes before the subject re-dressed in his clothing. The volunteer was allowed to carry out his normal indoor work activities for 30 minutes. After that period, the residual drug on the skin was recovered by wiping the application areas thoroughly with 3 alcohol pads. The pads were combined and extracted with ethanol to recover the ibuprofen. The concentration of ibuprofen in the extract was determined by the same HPLC method as described in EXAMPLE 7.

The amounts of ibuprofen recovered after 30 minutes are listed below:

| | Ibuprofen recovered |
|---|---|
| Test article/Location | in pads (mg) |
| F6 - Left-shoulder | 8.2 |
| IbuGel ® - Right-shoulder | 5.3 |
| F6 - Left-knee | 11.9 |
| IbuGel ® - Right-knee | 8.5 |

The F6 composition according to the present invention retained substantially more drug than the IbuGel®. This finding demonstrates the abrasion-resisting properties of the composition of the present invention, which make it less likely to be transferred to clothing or other body parts that can result in accidental secondary exposure.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A liquid composition for topical application, comprising: a polymethacrylate at a concentration greater than or equal to 5% of the composition weight, a humectant, a plasticizer, a solvent, and, optionally, a permeation enhancer and/or a drug, where said composition upon being sprayed or applied onto the skin is capable of rapidly forming a thin, surface-conforming, and skin-adherent film and delivering said drug transdermally, intradermally or maintaining it on the skin surface.

2. A composition according to claim 1, where the polymethacrylate is a methacrylic acid-ethyl acrylate copolymer.

3. A composition according to claim 2, wherein the polymethacrylate is present at a concentration between 5% and 20% of the composition weight, preferably between 6% and 18% of the composition weight, and more preferably between 10% and 16% of the composition weight.

4. A composition according to claim 1, where the humectant is selected from a group consisting of glycerol, sorbitol, propylene glycol, or a mixture thereof.

5. A composition according to claim 4, wherein the humectant is present at a concentration between 0.1% and 5% of the composition weight, preferably between 0.5% and 3% of the composition weight, and more preferably between 1% and 2% of the composition weight.

6. A composition according to claim 1, where the solvent is selected from a group consisting of water, ethanol, isopropanol, or a mixture thereof.

7. A composition according to claim 6, wherein the solvent is present at a concentration between 30% and 80% of the composition weight, preferably between 40% and 70% of the composition weight and more preferably between 50% and 60% of the composition weight.

8. A composition according to claim 1, where the permeation enhancer is selected from a group consisting of isopropyl myristate, camphor, geraniol, limonene, farnesol, eugenol, tween 20, tween 80, transcutol, DMSO, linalool, ethyl oleate, cineole and menthol, and a mixture thereof.

9. A composition according to claim 8, wherein the permeation enhancer is present at a concentration between 0.1% and 10% of the composition weight, preferably between 0.5% and 7% of the composition weight, and more preferably between 1% and 5% of the composition weight.

10. A composition according to claim 1, where the pH adjuster is selected from a group consisting of triethanolamine, meglumine, citric acid, and acetic acid.

11. A composition according to claim 10, wherein the pH adjuster is present at a concentration between 0.1% and 5% of the composition weight, preferably between 0.5% and 3% of the composition weight, and more preferably between 1% and 2% of the composition weight.

12. A composition according to claim 1, where the plasticizer is selected from a group consisting of triethyl citrate, dimethyl isosorbide, castor oil, propylene glycol, polyethylene glycol, or a mixture thereof.

13. A composition according to claim 12, wherein the plasticizer is present at a concentration between 0.1% and 5% of the composition weight, preferably between 0.5% and 3% of the composition weight, and more preferably between 1% and 2% of the composition weight.

14. A composition according to claim 1, where the composition contains no drug.

15. A composition according to claim 1, wherein the drug is present at a concentration between 0.1% and 25% of the composition weight, preferably between 0.5% and 20% of the composition weight, and more preferably between 1% and 15% of the composition weight.

16. A composition according to claim 1, where the composition has a viscosity of no more than 100 centipoises at room temperature and is sprayable.

17. A composition according to claim 1, where the composition, upon being sprayed or rubbed onto the skin, dries in no more than 5 minutes and forms a clear, skin-adherent and surface-conforming film that remains on the human skin for about 6 hours to 48 hours.

18. A composition according to claim 17, where the thickness of the film is between about 5 and 100 micrometers.

19. A composition according to claim 17, where the film delivers the drug transdermally, intradermally or maintains the drug on the skin surface.

20. A composition according to claim 17, where the film is non-smearing and does not transfer the drug to a new skin area or to other person by skin contact to cause the secondary exposure.

21. A composition according to claim 1, where the composition is used as a physical barrier to protect or treat the skin.

22. A composition according to claim 1, where the composition is applied to the skin, mucous membrane, nails and hairs of human and animals.

23. A composition according to claim 1, where the composition is used as a carrier for a cosmetic ingredient, scent, colorant, or personal care compound alone or in combination, including combinations having one or more drugs.
